(19) United States
(12) Patent Application Publication
(10) Pub. No.: US 2024/0293451 A1
(43) Pub. Date: Sep. 5, 2024

(54) ANGIOGENESIS MATERIAL, BONE-REGENERATION-PROMOTING MATERIAL, METHOD FOR PRODUCING ANGIOGENESIS MATERIAL, AND METHOD FOR PRODUCING BONE-REGENERATION-PROMOTING MATERIAL

(71) Applicant: TOHOKU UNIVERSITY, Sendai-shi (JP)

(72) Inventors: Osamu SUZUKI, Sendai-shi (JP); Ryo HAMAI, Sendai-shi (JP); Yukari SHIWAKU, Sendai-shi (JP); Kaori TSUCHIYA, Sendai-shi (JP); Shinki KOUYAMA, Sendai-shi (JP)

(21) Appl. No.: 18/659,445

(22) Filed: May 9, 2024

Related U.S. Application Data
(63) Continuation of application No. PCT/JP2022/042537, filed on Nov. 16, 2022.

(30) Foreign Application Priority Data
Nov. 17, 2021 (JP) 2021-187111

(51) Int. Cl. A61K 33/42; A61K 47/42; A61P 9/00; A61P 19/08

(52) U.S. Cl. CPC A61K 33/42; A61K 47/42; A61P 9/00; A61P 19/08

(57) ABSTRACT

An angiogenesis material, a bone-regeneration-promoting material, a method for producing an angiogenesis material and a method for producing a bone-regeneration-promoting material which particularly improve angiogenesis capacity, angiogenesis capacity and osteogenesis capacity, are particularly effective for bone regeneration and are expected to be applied not only to bone regeneration but also to regeneration of various tissues in which angiogenesis is required are provided. These are an angiogenesis material containing a composite of a metal-octacalcium phosphate-containing material containing octacalcium phosphate and a metal element and a bioabsorbable polymer, a bone-regeneration-promoting material using the same, and production methods thereof.

### CROSS-REFERENCE TO RELATED APPLICATIONS

This application is a continuation of International Application No. PCT/JP2022/042537, filed on Nov. 16, 2022, which, in turn, claims priority to Japanese Patent Application No. 2021-187111, filed on Nov. 17, 2021, both of which are hereby incorporated herein by reference in their entireties for all purposes.

### TECHNICAL FIELD

The present invention relates to an angiogenesis material, a bone-regeneration-promoting material using the same, and production methods thereof.

The present application claims priority of patent application No. 2021-187111 filed on Nov. 17, 2021 in Japan, and the contents thereof are incorporated herein.

### BACKGROUND ART

Bone grafting is generally used for patients with a bone defect following surgery of a bone tumor, cheilognathopalatoschisis, a comminuted fracture, or the like. Bone grafting is sometimes used also for repairing a bone defect resulting from surgery in the areas of brain surgery, orthopedics, dentistry, and the like.

For bone grafting, an autologous bone is preferably used. To use an autologous bone, however, there is a limit on the amount, and there is a problem such as the damage that remains after the autologous bone is taken out. Thus, an artificial bone which can replace the autologous bone has been developed as a bone used for bone grafting.

As artificial bone materials, hydroxyapatite (Ca₁₀ (PO₄)₆(OH)₂: sometimes referred to as “HA” below) ceramics, β-tricalcium phosphate (β-TCP) ceramics, and the like have been proposed.

It is known that a precursor of HA, octacalcium phosphate (Ca₈H₂ (PO₄)₆·5H₂O: sometimes referred to as “OCP” below), has many superior functions to those of HA. For example, octacalcium phosphate is excellent in osteoconductivity, absorbability by osteoclasts, and dose-dependent promotion of differentiation of osteoblasts. Moreover, it has been reported that precursors of HA, amorphous calcium phosphate (Ca₃ (PO₄)₂·nH₂O) and calcium hydrogen phosphate (anhydrous calcium hydrogen phosphate (CaHPO₄) or calcium hydrogen phosphate dihydrate (CaHPO₄·2H₂O)), have similar properties to those of OCP. Therefore, HA precursors such as OCP, rather than HA, are expected as promising artificial bone materials.

However, to use OCP as an artificial bone material, OCP is very fragile and has low formability. To compensate for the low formability of OCP, a composite of OCP and a polymer material has been examined. For example, a composite of granules of OCP and collagen (sometimes referred to as “OCP/Col” below) is known. Although OCP/Col promotes the excellent osteoconductivity of OCP, when OCP/Col is used as an artificial bone material, the rate of loss of the artificial bone is slower than the regeneration rate of the bone in the process in which the artificial bone is lost because the artificial bone component is absorbed by the surrounding tissue and in which the bone regenerates by replacing the artificial bone. This is a property caused because the granules of OCP are not easily absorbed completely in the living body. It is a very important issue required in this field that the regenerated bone sufficiently replaces the artificial bone and that the bone defect is completely filled with the new bone.

PTL 1 by the present inventors discloses a bone regeneration material containing a dehydrothermally cross-linked material of a co-precipitate of octacalcium phosphate and gelatin. PTL 1 also discloses a method for producing a bone regeneration material including a step of adding dropwise or injecting an aqueous solution containing calcium to an aqueous solution containing gelatin and phosphorus and thus obtaining a co-precipitate of octacalcium phosphate and gelatin and a step of heating the co-precipitate to obtain a dehydrothermally cross-linked material. With the technique, a bone regeneration material which has excellent physical strength and has the same shape as the shape of the bone before the defect and which can be sufficiently replaced by the new bone is to be provided using the co-precipitate of OCP and gelatin (OCP/Gel).

Moreover, NPL 1 by the present inventors has examined the bone regeneration using a composite obtained by mixing OCP granules in gelatin (OCP/Gel) as a bone regeneration material using rats and discloses that OCP/Gel contributes to angiogenesis. Angiogenesis is believed to promote bone regeneration through the blood flow of the surrounding tissue. OCP/Gel is excellent as a bone regeneration material due to the physical properties and the replacement with the new bone as described above but is shown to be suitable for bone regeneration also in view of the contribution to angiogenesis.

### CITATION LIST

### Patent Literature

PTL 1: JP2011-234799A

### Non Patent Literature

NPL 1: Suzuki et al., Acta Biomaterialia, 88(2019), 514-526

### SUMMARY OF INVENTION

### Technical Problem

In general, in bone regeneration, when angiogenesis is promoted, formation of a bone and replacement with a new bone are also promoted. Moreover, as NPL 1 shows, a bone regeneration material which has an aspect of contributing to angiogenesis is a material suitable for bone regeneration. However, there is no report on a material to which a potent function of further promoting angiogenesis is given or a bone-regeneration-promoting material which has both potent angiogenesis and bone-regeneration-promoting functions.

The invention has been made under the circumstances, and an object thereof is to provide an angiogenesis material, a bone-regeneration-promoting material, a method for producing an angiogenesis material and a method for producing a bone-regeneration-promoting material which particularly improve angiogenesis capacity, achieve both high angiogenesis capacity and osteogenesis capacity, and are particularly effective for bone regeneration.

### Solution to Problem

To solve the problem, the invention has the following aspects.

[1] An angiogenesis material containing a composite of a metal-octacalcium phosphate-containing material containing octacalcium phosphate and a metal element and a bioabsorbable polymer.

[2] The angiogenesis material, wherein the metal element is Cu or Zn.

[3] The angiogenesis material, wherein the bioabsorbable polymer is gelatin.

[4] A bone-regeneration-promoting material containing the angiogenesis material.

[5] A method for producing an angiogenesis material including mixing an aqueous solution containing phosphorus, an aqueous solution containing calcium, a solution containing a metal element and a bioabsorbable polymer, and thus obtaining an angiogenesis material containing a composite of a metal-octacalcium phosphate-containing material containing octacalcium phosphate and the metal element and the bioabsorbable polymer.

[6] The method for producing an angiogenesis material including a step of obtaining the metal-octacalcium phosphate-containing material containing octacalcium phosphate and the metal element by mixing the aqueous solution containing phosphorus, the aqueous solution containing calcium and the solution containing the metal element, and a step of obtaining the angiogenesis material which is the composite of the metal-octacalcium phosphate-containing material and the bioabsorbable polymer by mixing the metal-octacalcium phosphate-containing material and the bioabsorbable polymer.

[7] The method for producing an angiogenesis material, wherein, in the step of obtaining the metal-octacalcium phosphate-containing material, the metal-octacalcium phosphate-containing material is obtained by mixing the aqueous solution containing phosphorus, the aqueous solution containing calcium and the solution containing the metal element by dropwise addition or injection.

[8] The method for producing an angiogenesis material, wherein the metal element is Cu or Zn.

[9] The method for producing an angiogenesis material, wherein the bioabsorbable polymer is gelatin.

[10] A method for producing a bone-regeneration-promoting material including the step of obtaining the angiogenesis material in the method for producing an angiogenesis material, a wherein bone-regeneration-promoting material containing the angiogenesis material is obtained.

### Advantageous Effects of Invention

According to the invention, an angiogenesis material, a bone-regeneration-promoting material, a method for producing an angiogenesis material and a method for producing a bone-regeneration-promoting material which particularly improve angiogenesis capacity, achieve both high angiogenesis capacity and osteogenesis capacity and are particularly effective for bone regeneration can be provided.

### BRIEF DESCRIPTION OF DRAWINGS

FIG. 1 A graph figure showing the behaviors of Cu eluted from the Cu-OCP of the Examples.

FIG. 2 A graph figure of the low-Cu-OCP on another scale of the same test as that of FIG. 1.

### DESCRIPTION OF EMBODIMENTS

The angiogenesis material, the bone-regeneration-promoting material, and the production methods thereof according to the invention will be explained below with embodiments. The invention, however, is not limited to the following embodiments.

### (Angiogenesis Material)

The angiogenesis material of the embodiment contains a composite containing a metal-octacalcium phosphate-containing material (M-OCP) containing octacalcium phosphate (OCP) and a metal element and a bioabsorbable polymer.

The metal element can be appropriately selected, but in particular, a metal element which has no or small adverse influence on the biological tissues is preferably selected. In particular, a metal which can form a divalent metal ion is preferable. The metal element is preferably Cu or Zn. Cu or Zn is believed to be a metal element which is involved in angiogenesis. The metal element is particularly preferably Cu. Cu is involved in angiogenesis and is widely known as an essential mineral for organisms.

The composite containing M-OCP and a bioabsorbable polymer contains a metal element which promotes angiogenesis and thus exhibits the effect of particularly promoting regeneration of a bone due to the interaction of the physical and chemical properties suitable for bone regeneration that the conventional composite of OCP and a bioabsorbable polymer such as OCP/Gel has and the promotion of bone regeneration by angiogenesis in the tissue surrounding the regenerated bone.

The metal-octacalcium phosphate-containing material (M-OCP) may be in any form as long as the OCP and the metal element are combined. The metal element may exist in the form of an element, an ion, or a compound containing the metal element and an ion. The metal element or the ion may exist by partially substituting the calcium ions (Ca²⁺) in the crystal structure of OCP. The metal element and the OCP may be, for example, chemically bonded or may form a composite by physical interaction.

In the embodiment, copper (II) gluconate ([HOCH₂ (HCOH)₄COO]₂Cu, molecular weight of 453.85), copper (II) nitrate (Cu(NO₃)₂), copper (II) chloride (CuCl₃), copper (II) chloride dihydrate (CuCl₃·2H₂O), or copper (II) citrate (Cu₂ (C₆H₄O₇)) is used as the copper source. In view of the influence on the organism or the affinity for OCP, copper (II) gluconate is preferable as the copper source.

The ratio of the metal element and OCP contents in the M-OCP appropriately depends on the conditions for bone regeneration, but as a standard, for example, the metal element content, based on the total mass of the M-OCP, of the solid M-OCP before forming the composite with gelatin in the production method described below is preferably 0.001 to 0.50 wt %, particularly preferably 0.005 to 0.2 wt %, which can promote angiogenesis and bone regeneration without exhibiting cytotoxicity.

As the M-OCP, one produced by the production method described below can be used, and in addition, one produced using any means for adding the metal element in another conventionally known production method of OCP may also be used.

The bioabsorbable polymer of the embodiment is a polymer which can be absorbed in the living body. For example, a bioabsorbable natural polymer (gelatin, collagen, alginic acid, hyaluronic acid, chitosan, or the like) or a bioabsorbable synthetic polymer (polylactic acid, a polylactic acid-glycolic acid copolymer, polycaprolactone, or the like) can be widely used. The bioabsorbable polymer can be widely selected in the range in which the effects of the invention are not inhibited.

In the embodiment, gelatin is particularly preferably used as the bioabsorbable synthetic polymer.

The gelatin is not particularly limited. Generally, the gelatin is obtained by heat treating collagen. The gelatin may be commercial gelatin.

The collagen is not particularly limited. Examples thereof include collagen derived from porcine or bovine skin, bone or tendon. Enzyme-solubilized collagen which has been solubilized with a protease (for example, pepsin or pronase) and from which telopeptides have been removed is preferable. The type of the collagen is preferably, for example, type I or type I+type III. Collagen is a living body-derived component and is thus highly safe, and in particular, enzyme-solubilized collagen is preferable because the allergenicity is low. The collagen may be commercial collagen.

In the embodiment, when the gelatin is used, the angiogenesis material is a composite of M-OCP and gelatin (M-OCP/Gel). The M-OCP/Gel is a composite in which gelatin, which is modified collagen, and crystals of OCP containing the metal element are mixed, and the crystals of OCP containing the metal element are believed to be uniformly dispersed.

The phosphorus is not particularly limited as long as it is a compound which generates HPO₄²⁻ or PO₄³⁻ in an aqueous solution. Examples of such a compound include phosphates such as sodium hydrogen phosphate and ammonium phosphate and orthophosphoric acid.

The calcium is not particularly limited as long as it is a compound which generates Ca²⁺ in an aqueous solution. Examples of such a compound include calcium acetate, calcium chloride, and calcium nitrate.

The ratio of phosphorus and calcium is not particularly limited, but the ratio of phosphoric acid with calcium as 1, by mole ratio, is preferably 0.71 to 1.10, more preferably 0.73 to 1.00.

The ratio of the M-OCP and the bioabsorbable polymer is not particularly limited, but the ratio of the M-OCP with the bioabsorbable polymer as 1, by mass ratio, is preferably 0.1 to 9, more preferably 0.67 to 4. When the ratio of the M-OCP with the bioabsorbable polymer as 1 is less than 0.1, the bone regeneration capacity of the obtained bone regeneration material deteriorates, and when the ratio exceeds 9, the shape-imparting property decreases.

The angiogenesis material of the embodiment may contain a dehydrothermally cross-linked material of the composite of the M-OCP and the bioabsorbable polymer. The dehydrothermally cross-linked material is a structure in which the bioabsorbable polymer forming the composite are mutually cross-linked by a dehydrocondensation reaction. The dehydrothermally cross-linked material has a cross-linked structure and thus is physically highly strong and is absorbed in the living body at an adequate rate.

### (Bone-Regeneration-Promoting Material)

The bone-regeneration-promoting material of the embodiment contains the angiogenesis material described above. The bone-regeneration-promoting material may appropriately contain another component or may be appropriately processed so that the angiogenesis material described above is easily used for bone regeneration.

### (Production Method of Angiogenesis Material)

In the method for producing an angiogenesis material of the embodiment, an aqueous solution containing phosphoric acid, an aqueous solution containing calcium and a solution containing a metal element are mixed, and thus a metal-octacalcium phosphate-containing material containing the metal element is synthesized to obtain an angiogenesis material. Moreover, an angiogenesis regeneration material containing a composite obtained by mixing the metal-octacalcium phosphate-containing material containing octacalcium phosphate and the metal element and a bioabsorbable polymer is obtained.

The pH values of the aqueous solution containing phosphorus and the aqueous solution containing calcium are preferably 4.5 to 7.5. A buffer component may be contained to avoid a change in the pH through mixing of the aqueous solution containing calcium or the aqueous solution containing phosphorus. Moreover, the aqueous solution containing phosphorus, the aqueous solution containing calcium or the other solution may contain another component. The other component may contain a bioabsorbable polymer.

The solution containing the metal element is a solution containing the metal element described above or a compound containing the same. The solution containing the metal element may have similar pH as those of the aqueous solutions containing phosphorus or calcium described above, may contain a buffer component or the like, and may be added dropwise or injected under similar conditions. In the embodiment, an aqueous solution of copper (II) gluconate described above is used.

The bioabsorbable polymer can be selected from those listed above. In the embodiment, gelatin is used.

The production method of the embodiment preferably includes a step of obtaining a metal-octacalcium phosphate-containing material (M-OCP) containing octacalcium phosphate and the metal element by mixing the aqueous solution containing phosphorus, the aqueous solution containing calcium and the solution containing the metal element, and a step of obtaining an angiogenesis material which is a composite of the metal-octacalcium phosphate-containing material and the bioabsorbable polymer by mixing the metal-octacalcium phosphate-containing material and the bioabsorbable polymer.

Moreover, in the step of obtaining the metal-octacalcium phosphate-containing material, the metal-octacalcium phosphate-containing material is preferably obtained by mixing the aqueous solution containing phosphorus, the aqueous solution containing calcium, and mixing the solution containing the metal element by dropwise addition or injection.

The “dropwise addition” here means that droplets of a solution are added to another solution, and the “injection” means that a solution is added to another solution using a hollow tube such as a tube.

The dropwise addition or the injection may be conducted by adding dropwise or injecting another aqueous solution to any aqueous solution of the aqueous solution containing phosphorus, the aqueous solution containing calcium and the solution containing the metal element, or by adding dropwise or injecting another aqueous solution to another aqueous solution, such as a buffer.

The dropwise addition or the injection is conducted preferably at 50° C. to 80° C., more preferably at about 60° C. to 75° C. When the temperature is lower than 50° C. or exceeds 80° C., the M-OCP is not easily produced.

The dropwise addition or the injection is preferably conducted while stirring the aqueous solution. By stirring, the M-OCP obtained as granules described below has a uniform particle size.

The rate (mL/minute) of the dropwise addition or the injection is preferably 30 to 120, more preferably 35 to 82. When the rate is less than 30 or exceeds 120, the M-OCP is not easily produced.

The produced M-OCP is preferably also recovered as a solid. Examples of the solid include forms such as crystals and aggregates of crystals. Through the step of dropwise addition or injection, the M-OCP precipitates as crystals or aggregates of crystals and thus can be recovered from the aqueous solution by filtration, drying, or the like.

After preparation, the particle size of the M-OCP may be made uniform (sized). The particle size of the OCP granules obtained by sieving is preferably 10 to 1000 μm, more preferably 300 to 500 μm. As the sizing means, a filter or the like can be used.

The production method of the embodiment includes a step of recovering the metal-octacalcium phosphate-containing material (M-OCP), then mixing the bioabsorbable polymer and thus obtaining an angiogenesis material which is a composite of the metal-octacalcium phosphate-containing material and the bioabsorbable polymer. The bioabsorbable polymer may be, for example, an aqueous solution. Here, drying, freeze-drying, or the like may be used in the step of recovering the component of the composite of the M-OCP and the bioabsorbable polymer.

The M-OCP recovered as aggregates of crystals may be pulverized so that the composite with the bioabsorbable polymer is easily formed. The size after pulverization is preferably at nano size. The particle size is generally 5 to 1000 nm, preferably 100 to 400 nm. When the particle size exceeds 1000 nm, the absorbability of the bone regeneration material in the living body due to the physicochemical solubility of the Nano-OCP decreases. The pulverizing method is not particularly limited, but is preferably a method for mechanically pulverizing the OCP. The mechanically pulverizing means is not particularly limited. An example thereof is means using a hard tissue pulverizer (beads shocker), a ball mill, or a crusher.

In the production method of the embodiment, the aqueous phosphorus-containing solution and the aqueous calcium-containing solution which both or one of which contains the bioabsorbable polymer and the solution containing the metal element may be mixed by dropwise addition or injection to obtain a mixture of the metal-octacalcium phosphate-containing material and the bioabsorbable polymer, and the component of the composite of the M-OCP and the bioabsorbable polymer may be recovered directly using drying, freeze-drying, or the like.

In this regard, the production method of the embodiment may include a step of obtaining a cross-linked material by dehydrothermal cross-linking by cross-linking the bioabsorbable polymer in the composite through dehydrothermal condensation, chemical cross-linking by cross-linking through a covalent bond by a chemical reaction, or cross-linking by irradiation with electron beam or radiation. The dehydrothermally cross-linked material is obtained by heating the M-OCP/Gel. The heating treatment is conducted at a temperature of 50° C. to 200° C., preferably 100° C. to 150° C., for 3 hours to 240 hours, preferably 24 hours to 100 hours.

The dehydrothermally cross-linked material is preferably obtained by heating the composite of the M-OCP and the bioabsorbable polymer under reduced pressure. The reduced pressure condition is not particularly limited, but is, for example, 200 Pa or less, preferably 133 Pa or less.

The dehydrothermally cross-linked material is more preferably obtained by drying the composite of the M-OCP and the bioabsorbable polymer and then heating under reduced pressure. The drying method is not particularly limited, but is, for example, a freeze-drying method or a natural-drying method (air-drying). The drying step may be made efficient by leaving the composite of the M-OCP and the bioabsorbable polymer still before drying and then, for example, removing the supernatant to appropriately reduce the water content.

The angiogenesis material of the invention may contain a component which is generally contained in a bone regeneration material in the range in which the effects of the invention are not inhibited. Such a component is, for example, one selected from the bioabsorbable natural polymer used for the composite or another bioabsorbable natural polymer (gelatin, collagen, alginic acid, hyaluronic acid, chitosan, and the like), a bioabsorbable synthetic polymer (polylactic acid, a polylactic acid-glycolic acid copolymer, polycaprolactone, and the like), bioabsorbable calcium phosphate (β-TCP ceramics and the like), and a non-bioabsorbable material (HA ceramics and the like).

### (Production Method of Bone-Regeneration-Promoting Material)

The method for producing a bone-regeneration-promoting material of the embodiment includes the step of obtaining the angiogenesis material described above, and a bone-regeneration-promoting material containing the angiogenesis material is obtained. In the method for producing a bone-regeneration-promoting material, the angiogenesis material described above may be directly used for the bone-regeneration-promoting material, or the method may include a step of appropriately adding another component for easy use for bone regeneration or include a step of processing such as forming.

The bone-regeneration-promoting material of the invention is appropriately formed depending on the shape of the bone defect part and implanted in the bone defect part after sterilization treatment by irradiation with electron beam, high-pressure steam sterilization, or the like. The high-pressure steam sterilization, however, affects the crystal phase of the OCP, and thus in this case, the application site of the bone defect is taken into consideration.

Although embodiments of the invention have been explained above, the invention is not limited to the embodiments, and various changes can be made.

### EXAMPLES

The effects of the invention will be further clarified below with Examples and Comparative Examples. Here, the invention is not limited only to Examples below and can be carried out with an appropriate change in the range in which the gist thereof is not changed.

### (Synthesis of Cu-OCP/Gel)

### Example 1

By the wet method based on (Suzuki et al. Tohoku J. Exp. Med. 164 (1991) 37-50.), Cu-OCP using Cu as the metal element was synthesized as M-OCP.

An aqueous 0.04 mol/L calcium acetate solution in a volume of 1 L and an aqueous solution of copper (II) gluconate ([HOCH₂ (HCOH)₄COO]₂Cu, molecular weight of 453.85) as the Cu²⁺ source were mixed, and the mixture solution was mixed by adding dropwise over 15 minutes to 1 L of an aqueous phosphate solution (an aqueous 0.04 mol/L sodium dihydrogen phosphate dihydrate solution, pH 4.5 at room temperature). The concentration and the amount of the aqueous copper (II) gluconate solution added were in such a manner that the Cu content of the synthesized Cu-OCP granules as the charged amount of Cu element became approximately 0.014 wt % of the total mass of the Cu-OCP. The granules synthesized in Example 1 were named low-Cu-OCP.

After the completion of the dropwise addition, the mixture solution was further stirred at 65° C. for several minutes, and thus a precipitate (co-precipitate) was formed. The supernatant was removed, and the resultant was caused to pass through a 32 to 48 mesh filter to obtain granules of Cu-OCP (size of 300 to 500 μm).

Subsequently, an aqueous solution containing 3% (w/v) gelatin (manufactured by Sigma-Aldrich, Type A Gelatin (derived from porcine skin)) as a bioabsorbable polymer was prepared. The precipitate of Cu-OCP was added to the aqueous gelatin solution in such a manner that the Cu-OCP content became 44 wt % of the total mass of the Cu-OCP/Gel. The suspension of the precipitate was transferred to a polypropylene container, mixed at 4° C. for galating the gelatin, then frozen to −20° C. and freeze-dried, and thus an angiogenesis material containing a composite of M-OCP and gelatin (Cu-OCP/Gel) was obtained. The Cu-OCP/Gel was formed into a disk having a diameter of 9 mm and a thickness of 1 mm and vacuum heated at 150° C. for 24 hours to cross-link the gelatin. The material was used as the angiogenesis material for tests of angiogenesis and bone regeneration.

### Example 2

Cu-OCP was prepared in the same manner as in Example 1 except that copper (II) gluconate was added in such a manner that the Cu content of the synthesized M-OCP granules as the charged amount of Cu element became approximately 0.13 wt %. The granules synthesized in Example 2 were named high-Cu-OCP.

### Comparative Example 1

Preparation was conducted in the same manner as in Example 1 except that copper (II) gluconate was not added, which means that OCP was obtained instead of Cu-OCP.

### Comparative Example 2

Copper-containing gelatin (Cu/Gel) was prepared by the same method as that of Example 1 except that copper (II) gluconate was added instead of Cu-OCP or OCP. In this regard, copper (II) gluconate was added in such a manner that the copper content of the Cu/Gel became the same as the copper content of the low-Cu-OCP/Gel.

### (Cu Content of Cu-OCP)

The Cu-OCP in an amount of 20 mg was dissolved in 20 mL of 0.1 M HNO₃ during the preparation of Examples 1 and 2, and the Cu contents of the samples were measured using an inductively coupled plasma atomic emission spectroscopy (ICP-AES) device. The results are shown in Table 1. It was found that, regarding the Cu contents of the Cu-OCP, amounts corresponding to the aimed amounts of charged Cu in Examples 1 and 2 were contained.

TABLE 1

| | Amount of Charged Cu | Measured Cu Content |
|---|---|---|
| | (wt %) | (wt %) |
| Example 1 | 0.014 | 0.01 |
| Example 2 | 0.13 | 0.12 |

(in vitro Evaluation of Angiogenesis Capacity of Cu-OCP)

Human umbilical vein endothelial cells seeded on a 24-well plate coated with BD Matrigel™ Matrix (BD Biosciences, Bedford, MA, USA) were cultured for 24 hours in Endothelial Cell Growth Medium-2 medium in the presence of 1 mg of the OCP (Comparative Example 1), low-Cu-OCP (Example 1), or high-Cu-OCP (Example 2) granules. The culture under conditions without the granules was regarded as the control. After the culture, the shapes of the human umbilical vein endothelial cells were observed with an optical microscope. From the obtained optical microscopic images, the average values of the cross-linking points of the capillary-like structure formed by the human umbilical vein endothelial cells were measured.

The results of the comparison of the average values of the cross-linking points are shown in Table 2. The average values of the cross-linking points of the capillary-like structure formed by the human umbilical vein endothelial cells increased with the OCP (Comparative Example 1) and the low-Cu-OCP (Example 1) compared to the control. Moreover, the cross-linking points of the capillary-like structure particularly increased with the low-Cu-OCP (Example 1) compared to the OCP (Comparative Example 1) or the high-Cu-OCP (Example 2). Thus, it was considered that the amount corresponding to that of the low-Cu-OCP is particularly excellent as the Cu amount in the Cu-OCP and that the effect is particularly high when the Cu element content based on the total mass of the Cu-OCP is, for example, 0.005 to 0.1.

TABLE 2

| Conditions | Cross-Linking Points |
|---|---|
| Control | 92 |
| OCP | 108 |
| low-Cu-OCP | 137 |
| high-Cu-OCP | 75 |

### (In Vivo Evaluation of Angiogenesis Capacity and Osteogenesis Capacity of Cu-OCP/Gel)

A critical sized bone defect with a diameter of 9 mm was created in the calvaria of Wistar rats (male, 12 weeks old), and the OCP/Gel (Comparative Example 1), the Cu/Gel (Comparative Example 2) or the low-Cu-OCP/Gel composite (Example 1) formed into a diameter of 9 mm and a thickness of 1 mm was implanted. Two weeks or four weeks after the implantation, the calvaria tissue was collected after perfusion of a blood vessel contrast agent (Microfil®).

Before and after decalcification, μCT images of the calvaria tissue were taken, and the volumes of the calcified tissue and the new blood vessels were measured by analyzing the obtained images using 3D image analysis software.

Table 3 shows the volumes of the new blood vessels obtained using the contrast agent. The volume of the new blood vessels of the low-Cu-OCP/Gel (Example 1) was larger both after two weeks and after four weeks than those of the OCP/Gel (Comparative Example 1) or the Cu/Gel (Comparative Example 2). Moreover, a further increase in the volume of the new blood vessels was observed with the low-Cu-OCP/Gel after four weeks compared to that after two weeks.

TABLE 3

| Implanted | Implantation | |
|---|---|---|
| Material | Period (Week) | Volume (mm³) |
| Cu/Gel | 2 | 0.9 |
| OCP/Gel | 2 | 1.5 |
| low-Cu-OCP/Gel | 2 | 3.5 |
| Cu/Gel | 4 | 0.9 |
| OCP/Gel | 4 | 1.3 |
| low-Cu-OCP/Gel | 4 | 3.9 |

Table 4 shows the volumes of the calcified matter, namely the volumes of the newly formed bone. The volume of the Cu-OCP/Gel (Example 1) was larger than that of the OCP/Gel (Comparative Example 1) or the Cu/Gel (Comparative Example 2), and a further increase in the volume of the calcified matter was observed after four weeks compared to that after two weeks.

TABLE 4

| Implanted | Implantation | |
|---|---|---|
| Material | Period (Week) | Volume (mm³) |
| Cu/Gel | 2 | 10.3 |
| OCP/Gel | 2 | 40.4 |
| low-Cu-OCP/Gel | 2 | 53.9 |
| Cu/Gel | 4 | 13.9 |
| OCP/Gel | 4 | 50.8 |
| low-Cu-OCP/Gel | 4 | 60.0 |

From the results, it was shown that the bone regeneration material of the embodiment largely increases angiogenesis capacity and also promotes bone regeneration.

### (Evaluation of Cu Elution Behavior of Cu-OCP)

Low-Cu-OCP synthesized in the same manner as in Example 1 above and high-Cu-OCP synthesized in the same manner as in Example 2 were prepared each in an amount of 5 mg. The granule size was 300 to 500 μm. The granules each in an amount of 5 mg were immersed in 5 mL of a 150 mM tris hydroxymethyl aminomethane-hydrochloride (Tris-HCl) buffer (pH 7.4, 37° C.) and stirred by inverting.

The supernatants were recovered using centrifugation at elution times of 1, 3, 24, 72 or 168 hours after starting the immersion.

The Cu concentrations contained in the recovered supernatants were measured with an inductively coupled plasma emission spectrometer (analysis wavelength of 324.7 nm).

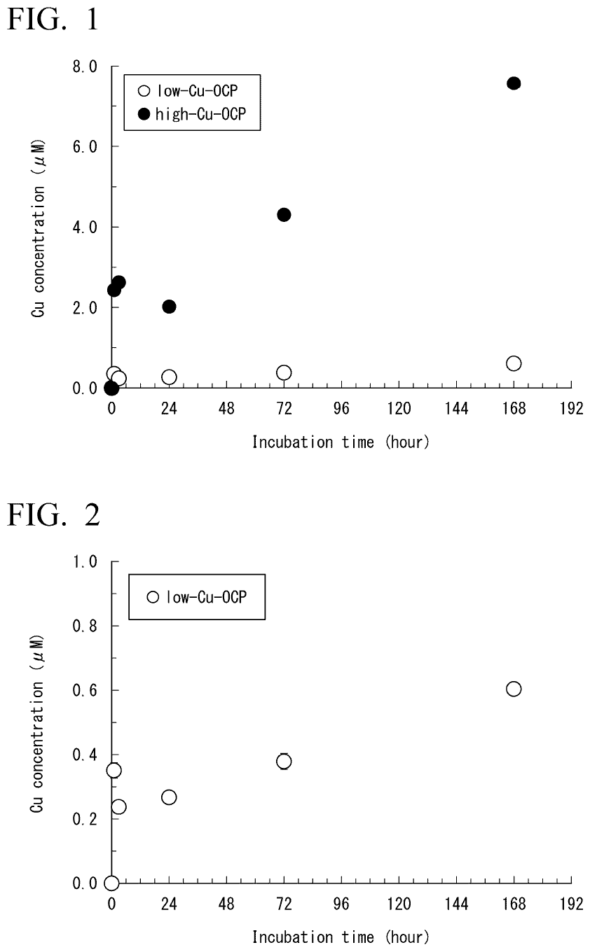

FIG. 1 shows the Cu concentrations contained in the supernatants at the elution times (incubation times). The concentrations of the low-Cu-OCP and the high-Cu-OCP synthesized in the same manner as in Example 2 are shown on the scale of 0 to 8.0 M.

FIG. 2 shows the concentrations of the low-Cu-OCP in the same measurement as in FIG. 1 on the scale of 0 to 1.0 UM.

As shown in FIGS. 1 and 2, the Cu concentration increased continuously at least to the elution time of 168 hours both from the low-Cu-OCP and the high-Cu-OCP. It was shown that Cu-OCP is a material capable of eluting Cu regardless of the Cu content. Moreover, because the Cu concentration from the low-Cu-OCP was lower than that from the high-Cu-OCP at each elution time, it was shown that a small amount of Cu is eluted continuously over a long time from granules of the low-Cu-OCP, and thus the low-Cu-OCP is less likely to cause cytotoxicity and promotes angiogenesis.

Furthermore, when the material of the embodiment is used for bone regeneration, because a bone is regenerated over a long time, it is believed to be effective that the effect of promoting angiogenesis lasts over a long time for bone regeneration due to the interaction with the angiogenesis effect of the embodiment.

### INDUSTRIAL APPLICABILITY

According to the angiogenesis material, the bone-regeneration-promoting material using the same, and the production methods thereof of the invention, an angiogenesis material having particularly improved angiogenesis capacity is obtained. Using the angiogenesis material for bone regeneration or as a bone-regeneration-promoting material, a bone-regeneration-promoting material which has both high angiogenesis capacity and osteogenesis capacity and which is particularly effective for bone regeneration and a production method thereof can be provided. Moreover, when angiogenesis capacity is focused, application not only to bone regeneration but also to regeneration of various tissues in which angiogenesis is required can be expected.

1. An angiogenesis material comprising a composite of a metal-octacalcium phosphate-containing material containing octacalcium phosphate and a metal element and a bioabsorbable polymer.

2. The angiogenesis material according to claim 1,
wherein the metal element is Cu or Zn.

3. The angiogenesis material according to claim 1,
wherein the bioabsorbable polymer is gelatin.

4. A bone-regeneration-promoting material comprising the angiogenesis material of claim 1.

5. A method for producing an angiogenesis material comprising:
mixing an aqueous solution containing phosphorus, an aqueous solution containing calcium, a solution containing a metal element, and a bioabsorbable polymer; and
thus obtaining an angiogenesis material containing a composite of a metal-octacalcium phosphate-containing material containing octacalcium phosphate and the metal element and the bioabsorbable polymer.

6. The method for producing an angiogenesis material according to claim 5, comprising:
a step of obtaining the metal-octacalcium phosphate-containing material containing octacalcium phosphate and the metal element by mixing the aqueous solution containing phosphorus, the aqueous solution containing calcium, and the solution containing the metal element; and
a step of obtaining the angiogenesis material which is the composite of the metal-octacalcium phosphate-containing material and the bioabsorbable polymer by mixing the metal-octacalcium phosphate-containing material and the bioabsorbable polymer.

7. The method for producing an angiogenesis material according to claim 6,
wherein, in the step of obtaining the metal-octacalcium phosphate-containing material, the metal-octacalcium phosphate-containing material is obtained by mixing the aqueous solution containing phosphorus, the aqueous solution containing calcium, and the solution containing the metal element by dropwise addition or injection.

8. The method for producing an angiogenesis material according to claim 5, wherein the metal element is Cu or Zn.

9. The method for producing an angiogenesis material according to claim 5, wherein the bioabsorbable polymer is gelatin.

10. A method for producing a bone-regeneration-promoting material comprising the step of obtaining the angiogenesis material in the method for producing an angiogenesis material according to claim 5, wherein a bone-regeneration-promoting material containing the angiogenesis material is obtained.
